(19) United States
(12) Patent Application Publication
(10) Pub. No.: US 2004/0063792 A1
(43) Pub. Date: Apr. 1, 2004

(54) Novel amorphous form of sertraline hydrochloride

(76) Inventors: Brij Khera, Princeton, NJ (US); Amit Rohatgi, Delhi (IN); Om Dutt Tyagi, Haryana (IN); Yatendra Kumar, Haryana (IN)

Correspondence Address: Jayadeep R Deshmukh, Ranbaxy Pharmaceuticals Inc, Suite 2100, 600 College Road East, Princeton, NY, 08540, US

(21) Appl. No.: 10/296,455

(22) PCT Filed: May 24, 2001
(86) PCT No.: PCT/IB01/00909

(30) Foreign Application Priority Data
May 26, 2000 (IN) 540/DEL/2000

(51) Int. Cl. A61K 31/137

(52) U.S. Cl. 514/649; 564/355

(57) ABSTRACT

This invention relates to a novel amorphous form of sertraline hydrochloride and a process for the preparation thereof.

### FIELD OF THE INVENTION

This invention relates to a novel amorphous form of sertraline hydrochloride and a process for the preparation thereof.

### BACKGROUND OF THE INVENTION

Sertraline hydrochloride is chemically, (1S-cis)4-(3,4-dichlorophenyl)-1,2,3,4-tetrahydro-N-methyl-1-naphthaleneamine hydrochloride and has the structural formula 1.

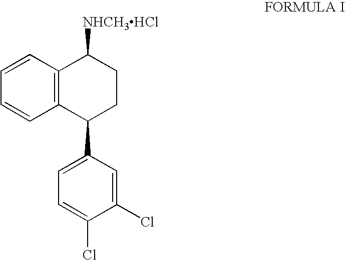

It is disclosed in U.S. Pat. No. 4,536,518, and is useful as an antidepressant and anorectic agent. It is also useful in treating conditions such as chemical dependencies and anxiety-related disorders.

The difference in the activity of different polymorphic forms of a given drug has drawn the attention of many workers in recent years. This has especially become very interesting after observing that many antibiotics, antibacterials, tranquilizers etc. exhibit polymorphism and some/one of the polymorphic forms of a given drug exhibit superior bioavailability and consequently show much higher activity compared to other polymorphs. The term polymorphism includes different physical forms, crystal forms, crystalline/ liquid crystalline/ non-crystalline (amorphous) forms.

It has also been disclosed that the amorphous forms in a number of drugs exhibit different dissolution characteristics and in some cases different bioavailability patterns compared to the crystalline form [Konne T., Chem. Pharm. Bull. 38, 2003 (1990)]. For some therapeutic indications one bioavailability pattern may be favoured over another. Cefuroxime axetil is the classical example of amorphous form exhibiting higher bioavailability than the crystalline form.

U.S. Pat. No. 5,248,699 discloses novel crystalline forms of sertraline hydrochloride, and reports five novel polymorphic forms, differing from one another in respect of their physical properties, stability, spectral data and methods of preparation. They are designated Form I, Form II, Form III, Form IV and Form V. The Form I product, however, is reported to have the greatest stability.

U.S. Pat. No. 5,734,083 discloses yet another crystalline polymorph, which is reported to exhibit improved bioavailability as compared to designated Form I sertraline hydrochloride. The said novel polymorph is designated polymorph T1.

### SUMMARY OF THE INVENTION

It is an objective of the present invention to provide a new amorphous form of sertraline hydrochloride and a process for the preparation thereof. The present process uses conditions which are convenient to perform on a commercial scale and operationally safe.

Accordingly, the present invention provides an amorphous form of sertraline hydrochloride and a process for the preparation thereof. The process comprises dissolving crystalline sertraline hydrochloride in a suitable solvent(s) or dissolving sertraline base in a suitable solvent(s) and adding a suitable solvent(s) containing hydrogen chloride and recovering amorphous form of sertraline hydrochloride from the solution thereof by the removal of solvent by a conventional technique. Such conventional techniques include, but are not limited to, distillation, distillation under vacuum, evaporation, spray drying, freeze drying, etc.

In a preferred embodiment of the invention, sertraline hydrochloride is recovered from the solution in an amorphous form using a freeze drying technique. The freeze dryer (Model : Virtis Genesis SQ Freeze Dryer), which is used operates on the principle of lyophilization i.e. a process of stabilizing initially wet materials (aqueous solution or suspensions) by freezing them, then subliming the ice while simultaneously desorbing some of the bound moisture (primary drying). Following disappearance of the ice, desorption may be prolonged (secondary drying). This process is usually conducted under vacuum.

In a more preferred embodiment of the invention, sertraline hydro-chloride is recovered from the solution in an amorphous form using a spray drying technique. The Mini-Spray Dryer (Model: Buchi 190, Switzerland) which is used, operates on the principle of nozzle spraying in a parallel flow, i.e., the sprayed product and the drying gas flow in the same direction. The drying gas can be air or inert gases such as nitrogen, argon and carbon dioxide. Nitrogen is preferred in this case.

The term “suitable solvent” means lower alkanol, ketones, esters, chlorinated solvents, acetonitrile or mixtures thereof, optionally in the presence of water. Lower alkanol includes those primary, secondary and tertiary alcohols having from one to six carbon atoms. Suitable lower alkanol solvents include methanol, ethanol, denatured spirit, n-propanol, isopropanol, n-butanol, isobutanol and t-butanol. The term ketones or esters include solvents such as acetone, 2-butanone, 4-methylpentan-2-one, ethyl acetate and n-butylacetate. The suitable chlorinated solvents include dichloromethane and chloroform. Mixtures of these solvents are also contemplated.

Hydrogen chloride may be used either in the anhydrous gaseous form which is absorbed in the said suitable solvent(s) or an aqueous solution of hydrochloric acid may also be used. In general, molar equivalent proportions of hydrogen chloride and sertraline base should be used but varying amounts of molar concentrations are within the scope of this invention.

Methods known in the art may be used with the process of this invention to enhance any aspect of this process. For example, the product obtained may further be dried to achieve the desired moisture values. It may be dried in a tray drier or dried under vacuum or in a Fluid Bed Dryer.

The transition temperature for the conversion of the amorphous form of sertraline hydrochloride to its crystalline form appears to be low. Accordingly, due caution must be taken to keep the vacuum oven temperatures of below 40° C.

### BRIEF DESCRIPTION OF THE DRAWINGS

FIG. 1 shows the infra-red spectrum in KBr of the amorphous sertraline hydrochloride of the present invention.

FIG. 2 shows the x-ray powder diffraction pattern of the amorphous sertraline hydrochloride of the present invention.

FIG. 3 shows the infra-red spectrum in KBr for crystalline form, designated Form I of sertraline hydrochloride obtained per U.S. Patent No. 5,248,699.

FIG. 4 shows the x-ray powder diffraction patterns obtained for the samples of a crystalline sertraline hydrochloride obtained per U.S. Patent No. 5,248,699.

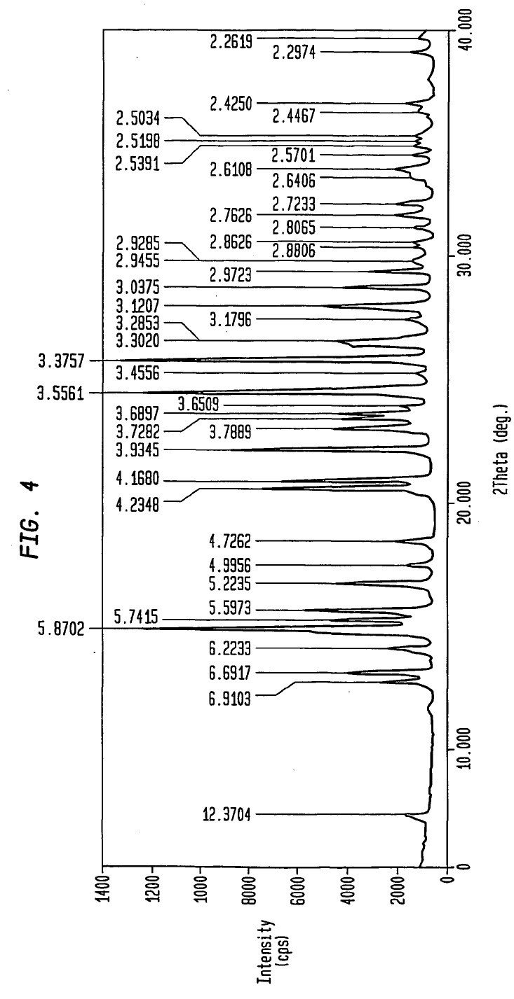

### DETAILED DESCRIPTION OF THE INVENTION

The present invention is illustrated by the following examples, which are not intended to limit the effective scope of this invention in any way.

Preparation of amorphous sertraline hydrochloride from crystalline sertraline hydrochloride.

### EXAMPLE 1

Sertraline hydrochloride crystalline (25g) was dissolved in methanol (400ml) at 48-52° C. The resulting clear solution was then cooled to an ambient temperature (30° C.) and subjected to spray drying in a Mini-Spray Dryer (Buchi Model - 190) at an inlet temperature 89-91° C. and outlet temperature 61-42° C. using nitrogen gas. The snow-white fine powder of sertraline hydrochloride in an amorphous form was collected. It was further dried for 12 hours under reduced pressure at a temperature not exceeding 40° C. to yield 16g of the desired product, having a purity of 99.8% w/w (by titrimetric analysis) and total impurities 0.43% w/w (by HPLC).

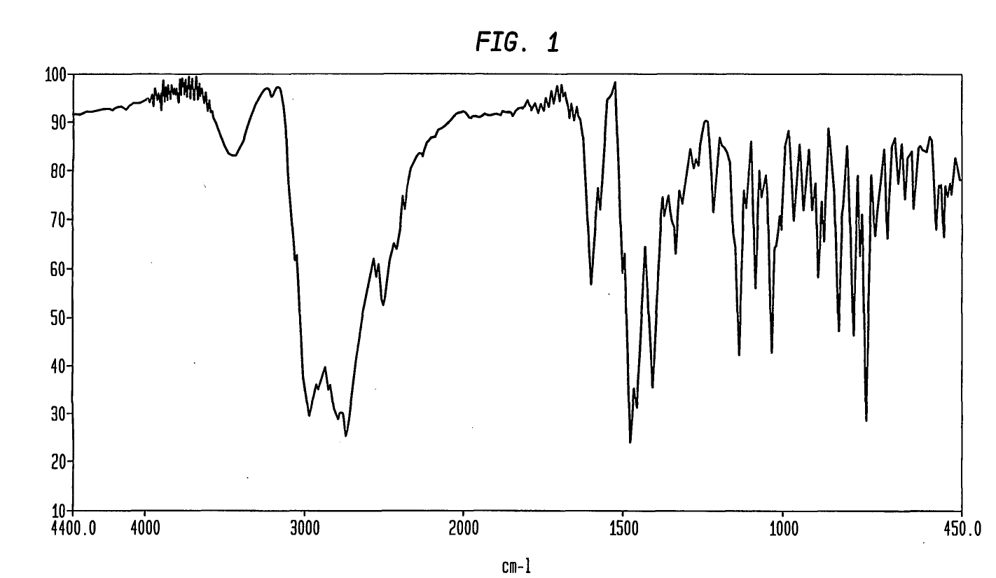

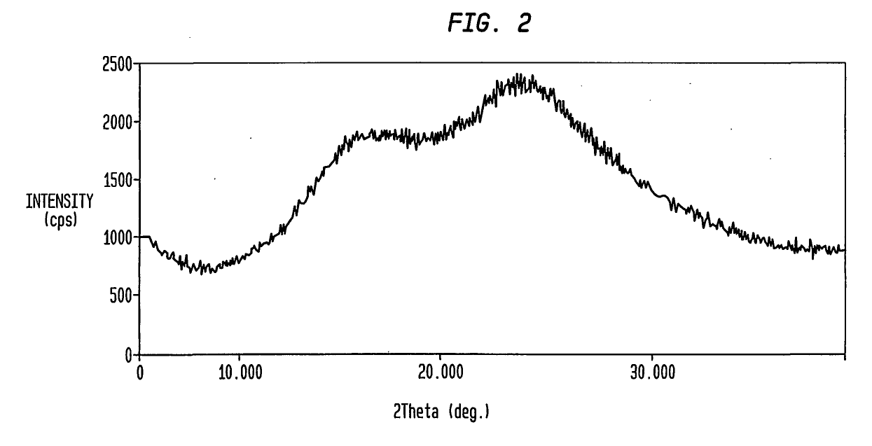

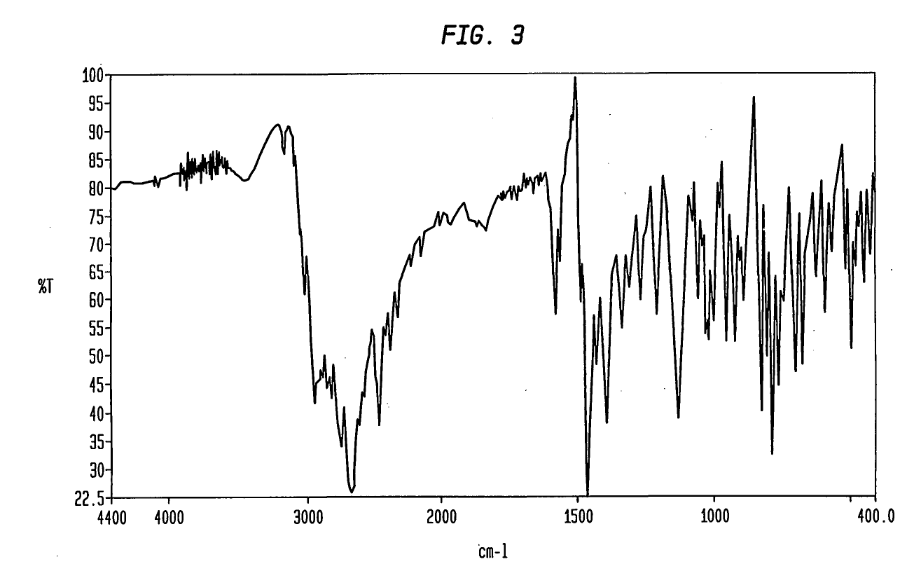

X-ray powder diffraction pattern (FIG. 2) does not exhibit any peak and shows a plain halo thus demonstrating the amorphous nature of the product. Infrared spectrum in KBr (FIG. 1) is different than the one obtained for crystalline form of sertraline hydrochloride (FIG. 3).

### EXAMPLE 2

Sertraline hydrochloride crystalline (125g) was dissolved in denatured spirit [DNS) (1.25 Lt) at 45-50° C. The resulting clear solution was subjected to spray drying in a Mini-Spray Dryer (Buchi Model -190) at an inlet temperature 90-100° C. and outlet temperature 60-43° C. using nitrogen gas. The snow-white fine powder of sertraline hydrochloride in an amorphous form was collected. It was further dried for 10 hours under reduced pressure at a temperature not exceeding 30° C. to yield 110 g of the desired product, having a purity of 99.4% w/w (by titrimetric analysis) and total impurities 0.569% w/w (by HPLC).

### EXAMPLE 3

Sertraline hydrochloride crystalline (50g) was dissolved in a mixture of acetone (300ml) and demineralized water (60ml) at 45-50° C. The resulting clear solution was subjected to spray drying in a Mini-Spray Dryer (Buchi Model -190) at an inlet temperature 97-99° C. and outlet temperature 52-48° C. using nitrogen gas. The snow-white fine powder of sertraline hydrochloride in an amorphous form was collected. It was further dried for 12 hours under reduced pressure at a temperature not exceeding 30° C. to yield 40g of the desired product. The product was found to be amorphous.

While the present invention has been described in terms of its specific embodiments, certain. modifications and equivalents will be apparent to those skilled in the art and are intended to be included within the scope of the present invention.

We claim:

1. Amorphous sertraline hydrochloride.

2. A process for the preparation of sertraline hydrochloride in amorphous form which comprises dissolving crystalline sertraline hydrochloride in suitable solvent(s) or dissolving sertraline base in suitable solvent(s) and adding suitable solvent(s) containing hydrogen chloride and recovering sertraline hydrochloride in the amorphous form from the solution thereof by the removal of the solvent.

3. The process of claim 2 wherein suitable solvent means lower alkanol, ketone, ester, chlorinated solvent, acetonitrile or mixtures thereof, optionally in the presence of water.

4. The process of claim 3 wherein lower alkanol includes primary, secondary and tertiary alcohol's having from one to six carbon atoms.

5. The process of claim 4 wherein the said lower alkanol is selected from the group consisting of methanol, ethanol, denatured spirit, n-propanol, isopropanol, n-butanol, isobutanot, t-butanol or mixtures thereof.

6. The process of claim 5 wherein the preferred solvent is methanol, ethanol or denatured spirit.

7. The process of claim 3 wherein ketone is acetone, 2-butanone, 4-methylpentan-2-one or mixtures thereof.

8. The process of claim 3 wherein ester is selected from ethyl acetate or n-butyl acetate or mixtures thereof.

9. The process of claim 3 wherein chlorinated solvent is chloroform, dichloromethane or mixtures thereof.

10. The process of claim 2 wherein hydrogen chloride is either anhydrous and present in the gaseous form absorbed in the said suitable solvent or an aqueous solution of hydrochloric acid.

11. The process of claim 10 wherein hydrogen chloride is present in equimolar amounts.

12. The process of claim 2 wherein the solvent is removed by a conventional technique.

13. The process of claim 2 wherein the conventional technique includes distillation, distillation under vacuum, evaporation, spray drying, or freeze drying.

14. The process of claim 2 wherein sertraline hydrochloride in an amorphous form is recovered from the said solution by spray drying.

15. The process of claim 14 wherein the spray drying is effected in the presence of an inert gas.

16. The process of claim 2 wherein sertraline hydrochloride in an amorphous form is recovered from the said solution by freeze-drying.

17. The process of claim 2 wherein the product obtained is further dried.

18. The process of claim 17 wherein the drying is carried out below 40° C.
